(19) United States
(12) Patent Application Publication
(10) Pub. No.: US 2007/0092923 A1
(43) Pub. Date: Apr. 26, 2007

(54) ESTABLISHING A LONG-TERM PROFILE OF BLOOD SUGAR LEVEL AIDING SELF-CONTROL OF THE SAME

(76) Inventor: Chin-Lien Chang, Hsin-Chu City (TW)

Correspondence Address: BERKELEY LAW & TECHNOLOGY GROUP, LLP, 1700 NW 167TH PLACE, SUITE 240, BEAVERTON, OR, 97006, US

(21) Appl. No.: 11/469,402

(22) Filed: Aug. 31, 2006

Related U.S. Application Data
(62) Division of application No. 10/316,049, filed on Dec. 11, 2002.

(51) Int. Cl. C12Q 1/54; G06F 19/00

(52) U.S. Cl. 435/14; 702/19

(57) ABSTRACT

Establishing a long-term profile of blood sugar level aiding self-control of the same includes periodically obtaining a specimen from a patient, and detecting a response of the specimen. Obtaining a blood sugar value of the patient based on the response, and storing the blood sugar values periodically measured. Establishing a long-term profile of blood sugar level from the measured blood sugar values in a form of chart. Furthermore, when the variation of the long-term profile of blood sugar level exceeds a standard, generating a warning signal informing the patient. When the variation of the long-term profile of blood sugar level does not exceed the standard, generating a safety signal informing the patient.

### BACKGROUND OF THE INVENTION

1. Field of the Invention

The present invention relates to a method for establishing a long-term profile of the concentration of a specific component of blood, and in particular, to a method for establishing a long-term profile of the concentration of glucose of blood aiding a patient to self-control blood sugar level.

2. Description of the Prior Art

There are 11 million known diabetics in the United States. It is estimated that another 20 million people are predisposed to this disease while as many as 10 million diabetics may remain undiagnosed. Diabetes is a relentless disease constantly degrading the person's health. Obesity is considered a major contributor to the disease rather than a symptom of it. Insulin from the body normally controls the level of blood sugar. However, in type 2 diabetes, the body's insulin is not effective and blood sugar levels rise too high. Complications of this effect can include kidney disease, blindness, and gangrene, which can require leg and foot amputations.

It has been said that no one ever dies of diabetes. It is common knowledge, however, that these persons live short lives with a multitude of health complications. Because control of this disease is an hour by hour health control problem for the diabetics, they must be made an informed partner in the maintenance of their health. This means that control of the disease is the primarily responsibility of the diabetic patient with minimal supervision by health care specialists.

The self-control of the blood sugar level is achieved through a diet, exercise and medication. These treatments may often be simultaneously employed under supervision of the doctor. It has been found that the self-control works more effectively when the patient himself is able to check whether or not this blood sugar level is within the normal range.

Recently, blood sugar determining instruments have been used for self-checking of blood sugar level. As shown in FIG. 1, a blood sugar determining instrument mainly includes a main detecting unit 10 and a chip 12 for blood sugar measurement. As shown in FIG. 2, the chip 12 includes a strip-like substrate 122 provided in its front portion with an electrode section 1221. The electrode section 1221 is covered by a reaction layer 124, a spacer 126 and a cover sheet 128. The electrode section 1221 is provided an operational terminal 1222 and a counterpart terminal 1224 surrounding the operational terminal 1222. The operational terminal 1222 and the counterpart terminal 1224 are electrically connected to lead terminals 1226 and 1228, respectively, which are formed on a base end portion of the substrate 122. The reaction layer 124, which covers the electrode section 1221, contains potassium ferricyanide and an oxidase-such as glucose oxidase.

The blood sugar determining instrument may be used in the following manner. A patient pricks his or her own skin with e.g. a lancet for oozing blood. Then, the oozed-out blood is caused to touch the tip of the chip 12 plugged into the detecting unit 10. The blood is partially sucked into the reaction layer 124, disposed above the electrode section 1221, is dissolved by the blood, which starts an elementary reaction.

The potassium ferricyanide contained in the reaction layer 124 is reduced, whereas potassium ferrocyanide or reduced electron carrier is accumulated. The amount of the potassium ferrocyanide is proportional to the concentration of glucose to be measured. When the potassium ferrocyanide accumulated for a specific time is electrochemically oxidized by application of a certain voltage, a response current will pass through the operational terminal 1222. Thus, the glucose concentration (blood sugar level) is determined by measuring the response current with the detecting unit 10. The detected current is converted into a glucose concentration value by the detecting unit 10 as output reading of a blood sugar value.

Diabetes patients often measure the blood sugar level two, three, four, or five times a day. The conventional blood sugar determining instrument can store 20 to 50 readings of the measured blood sugar levels one time. However, the conventional blood sugar determining instrument can display only one reading one time, and not output a report for all the measured blood sugar levels. Therefore, patients typically take their own blood sugar level readings using the conventional blood sugar determining instrument, and record the readings by writing the result on a chart. The patient can not view continuous variation of the blood sugar level in his/her body from a long-term profile of blood sugar level. It is very inconvenient for the patient desires to check the variation of the blood sugar level for each day or a period of time to self-control the blood sugar level in his/her body through the diet and medication. Moreover, medical practitioners, who are increasingly working with more patients with fewer time devoted to each patient, simply do not have time to carefully review a stack of hand-written record sheets to determine whether a patient's recent readings are significantly improved in comparison to the patient's previous readings. As a result, the diagnosis and treatment of diabetes may be delayed and inaccurate.

Accordingly, it is an intention to provide a method enabling to facilitate establishing a long-term profile of blood sugar level associated with a blood sugar determining instrument to resolve the above concerns.

### SUMMARY OF THE INVENTION

It is one objective of the present invention to provide a method for establishing a long-term profile of blood sugar level for a patient to make the self-control of the blood sugar level works more effectively.

It is another objective of the present invention to provide a method for establishing a long-term profile of blood sugar level to help a patient control the concentration of glucose in his/her body through a diet, exercise and medication.

It is further an objective of the present invention to provide a method for establishing a long-term profile of blood sugar level for a patient to facilitate the diagnosis and treatment of the diabetes.

It is still a further objective of the present invention to provide a method for establishing a long-term profile of blood sugar level, which can directly download the measured blood sugar values in a computer system to facilitate establishing a long-term profile of the blood sugar level for a patient, and thus aiding the patient self-control the blood sugar level quicker and more accurate.

In order to achieve the above objectives of this invention, the present invention provides a method for establishing a long-term profile of blood sugar level aiding self-control of the same. The present invention includes periodically obtaining a specimen from a patient and detecting a response of the specimen. Obtaining a blood sugar value of the patient based on the response, and storing the blood sugar values periodically measured. Then, displaying the blood sugar values in a chart so as to establish a long-term profile of blood sugar level for the patient. The present invention further comprising judging the variation of the long-term profile of blood sugar level, when the variation of the long-term profile of blood sugar level exceeds a standard, generating a warning signal informing the patient. On the contrary, when the variation of the long-term profile of blood sugar level does not exceed the standard, generating a safety signal informing the patient. The measured blood sugar values can be stored in a memory of a blood sugar determining instrument used for detecting the blood samples, and directly displayed in the form of chart on a panel of the blood sugar determining instrument. The measured blood sugar values stored in the memory of the blood sugar determining instrument also can be directly download in a computer system to facilitate establishing the long-term profile of blood sugar level, and displaying the long-term profile of blood sugar level on a window of the computer system. The present invention provides a method for establishing a long-term profile of blood sugar level quicker and more accurate, which is able to help a patient more effectively self control the blood sugar level in his/her body through a diet, exercise and medication. The diagnosis and treatment of diabetes also can be facilitated.

### BRIEF DESCRIPTION OF THE DRAWINGS

The objectives and features of the present invention as well as advantages thereof will become apparent from the following detailed description, considered in conjunction with the accompanying drawings.

FIG. 1 is a perspective view showing an external appearance of a conventional blood sugar determining instrument;

FIG. 2 is an enlarged exploded view showing the chip used for the conventional blood sugar determining instrument;

FIG. 3 is a flow chart of the present method according to a first embodiment of the present invention; and

FIG. 4 is a flow chart of the present method according to a second embodiment of the present invention.

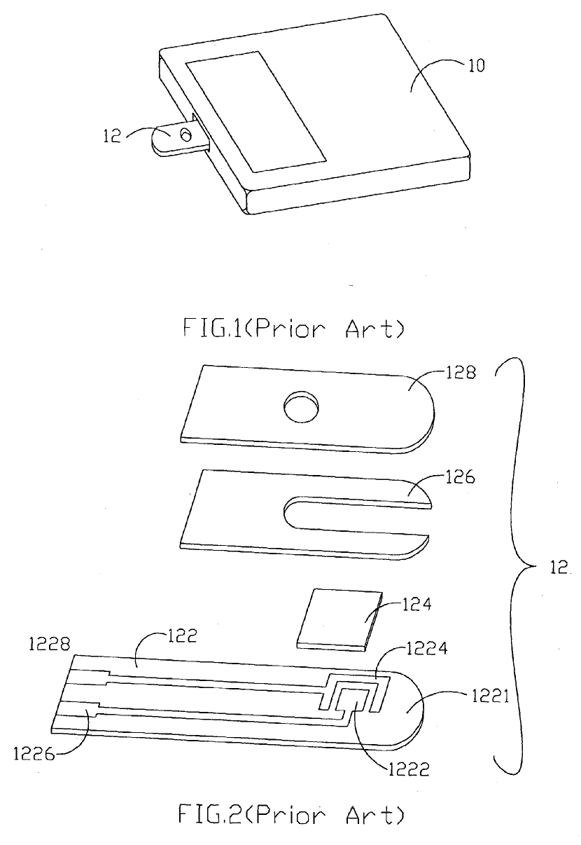

### DESCRIPTION OF THE EMBODIMENTS

The present invention provides a method for establishing a long-term profile of blood sugar level aiding a patient to self-control the concentration of glucose in his/her body through a diet, exercise and medication. The present method includes periodically sampling a blood sample from a patient, and detecting a response of the blood sample. Next, determining a blood sugar value of the blood sample based on the response. Storing the blood sugar values periodically measured, and displaying the blood sugar values in a form of chart so as to establish a long-term profile of blood sugar level for the patient. Then, judging the variation of the long-term profile of blood sugar level. When the variation of the long-term profile of blood sugar level exceeds a standard, generating a warning signal informing the patient, and when the variation of the long-term profile of blood sugar level does not exceed the standard, generating a safety signal informing the patient.

On the other hand, the present invention is not limited to measure the blood sugar level of the patient from the blood sample. The present invention also can use a blood sugar determining instrument to directly contact a specific portion of the body of the patient to obtain a response corresponding to the blood sugar level in his/her body. Alternately, the present invention can use a blood sugar determining instrument to directly contact a specific portion of the body of the patient, and obtaining a response corresponding to the blood sugar level in his/her body via the sweat of the patient. As a consequence, the patient would not suffer from the pain due to sampling the blood sample from his/her body.

The measured blood sugar values can be stored in a memory of the blood sugar determining instrument, and then displayed on a panel of the blood sugar determining instrument in the form of chart. Therefore, a long-term profile of blood sugar level established by the measured blood sugar values is provided on the panel of the blood sugar determining instrument. Alternately, the measured blood sugar values can be directly download in a computer system from the blood sugar determining instrument via an interface, and then displayed on a window of the computer system in the form of chart by running a graphic software installed in the computer system. Thus, a long-term profile of the blood sugar level can be provided on the window of the computer system. The present method facilitates establishment of the long-term profile of blood sugar level. It is convenient for the patient to self-check the variation of the blood sugar level in his/her body because the long-term profile of blood sugar level is directly displayed on the panel of the blood sugar determining instrument or the window of the computer system communicating with the blood sugar determining instrument. By the way, the present method provides a reminder e.g. a warning or safety signal, in accordance with the variation of the long-term profile of blood sugar level, to inform the patient whether his/her blood sugar level is within the normal range or not, which makes the self-control of the blood sugar level of the patient works more effectively and accurate. The diagnosis and treatment of diabetes also can be improved.

The present invention will be described in detail in accordance with the following embodiments with reference to accompanying drawings.

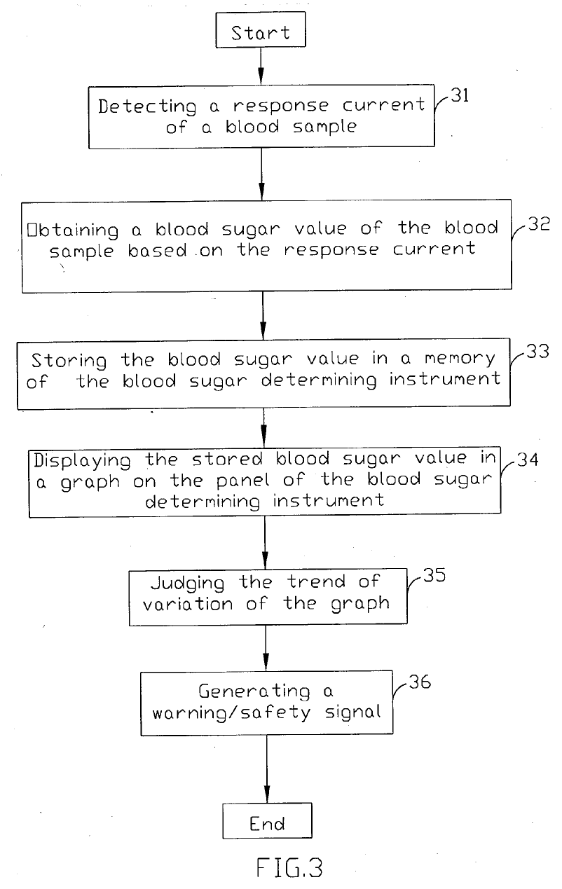

FIG. 3 is a flow chart of the present method for establishing a long-term profile of blood sugar level in accordance with a first embodiment of the present invention. The flow chart of the present method of the first embodiment begins at step 31, periodically sampling a blood sample of a patient, for example pricking the patient's skin to ooze blood for sampling. And, the sampling can be two times, three times, or five times one day, or before each mealtime, for example, before breakfast, lunch and dinner. The sampling can be continued for a period of time such as one week, one month, three months, six months, etc. The oozing-out blood sample is dropped in a reaction zone of a blood sugar determining instrument used for measuring the concentration of glucose, i.e. the blood sugar level, in the blood sample. A well-known method to measure the blood sugar level is utilizing a specific enzyme catalytic reaction with the blood sample proceeding in the reaction zone, and detecting a current response generated thereby when applying a voltage on the reaction zone. The current response is proportional to the concentration of the glucose in the blood sample. Therefore, go to step 32, determining the blood sugar value of the blood sample in accordance with the current response, i.e. the current response is converted to the blood sugar value by the blood sugar determining instrument.

Next, go to step 33, storing the measured blood sugar value in a memory of the blood sugar determining instrument. Then, at step 34, the measured blood sugar value and previous measured blood sugar values having been stored in the memory are simultaneously displayed on a panel of the blood sugar determining instrument in a form of chart of glucose concentration verse sampling time, such as a strip chart, a curve, or a table list, in order that a long-term profile of blood sugar level for the patient established by all the measured blood sugar values is provided on the panel of the blood sugar determining instrument. Go to step 35, judging the variation of the long-term profile of blood sugar level. In case that the long-term profile of blood sugar level is in the form of a curve established by all the measured blood sugar values, the change of the slope of the curve can be used as a reference to determine whether the blood sugar level of the patient trends toward high levels. Alternately, when the long-term profile of blood sugar level is in the form of strip chart, the frequency of the strip lines each of which representing a measured blood sugar value exceeding a normal value can be used as a reference to determine whether the blood sugar level of the patient trends toward high levels. When the long-term profile of blood sugar level is in the form of a table list with the measured blood sugar values exceeding a normal value having been marked or highlighted, the frequency of the measured blood sugar values being marked or highlighted can be used as a reference to determine whether the blood sugar level of the patient trends toward high levels. By viewing the long-term profile of blood sugar level establishing by the accumulated measured blood sugar values, the patient can self-check whether or not the blood sugar level in his/her body is too high upon the time for measuring the blood sugar level, or trends toward high levels during the period of time for measuring the blood sugar levels.

Then, go to step 36, when the variation of the long-term profile of blood sugar level exceeds a standard, the blood sugar determining instrument generates a warning signal to inform the patient to improve medication or take care his/her diet. As mentioned in step 35, the variation of the long-term profile of blood sugar level depends upon e.g. the change of the slope of the curve, the frequency of the strip lines of the strip chart each of which representing a measured blood sugar value exceeding the normal value, and the frequency of the blood sugar values having been marked or highlighted in the table list. When the variation of the long-term profile of blood sugar level does not exceed a standard, the blood sugar determining instrument generates a safety signal to inform the patient his/her blood sugar level is within the normal range. The warning/safety signal can be outputted in a form of voice, pattern and text string.

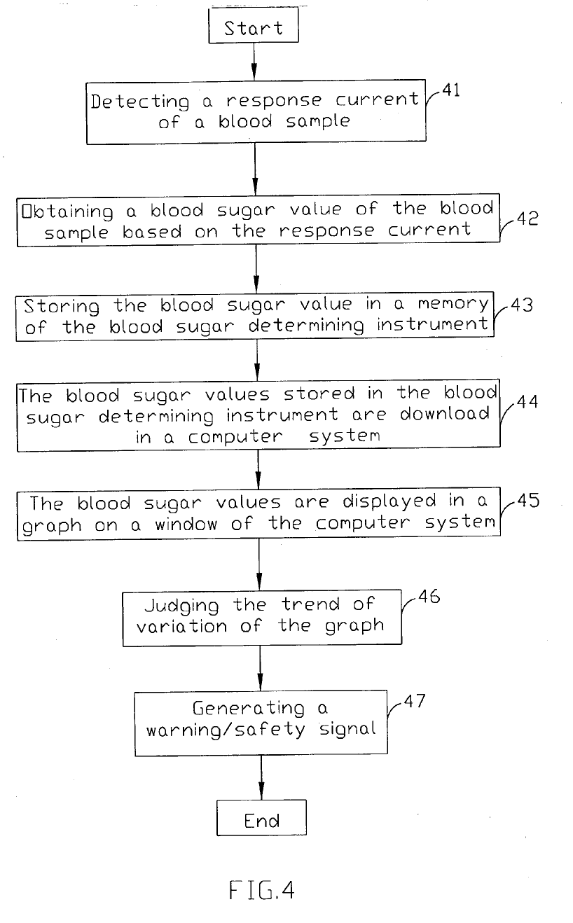

FIG. 4 is a flow chart of the present method according to a second embodiment of the present invention. It should be noted that step 41 to step 43 of the present method of the second embodiment are the same with the step 31 to step 33 of the first embodiment. While, at step 44 of the second embodiment, the blood sugar values stored in the memory of the blood sugar determining instrument are directly downloaded in a computer system via an interface. For example, the blood sugar determining instrument can be provided with a USB (Universal Serial Bus) connector to communicate with an USB interface incorporated in the computer system. The blood sugar values stored in the memory of the blood sugar determining instrument thus can be downloaded in the computer system passing through the USB interface. The computer system can be a personal computer, a notebook and a palm digital assistant (PDA). Go to step 45, the blood sugar values are simultaneously displayed on a window of the computer system in a form of chart, such as a strip chart, a curve, or a table list, by running a graphic software installed in the computer system. As a result, a long-term profile of blood sugar level established by all the measured blood sugar values can be provided on the window of the computer system, facilitating the self-checking of the patient. Go to step 46, judging the variation of the long-term profile of blood sugar level. In case that the long-term profile of blood sugar level is in the form of a curve established by all the measured blood sugar values, the change of the slope of the curve can be used as a reference to determine whether the blood sugar level of the patient trends toward high levels. Alternately, when the long-term profile of blood sugar level is in the form of strip chart, the frequency of the strip lines each of which representing a measured blood sugar value exceeding a normal value can be used as a reference to determine whether the blood sugar level of the patient trends toward high levels. When the long-term profile of blood sugar level is in the form of a table list with the measured blood sugar values exceeding a normal value having been marked or highlighted, the frequency of the measured blood sugar values being marked or highlighted can be used as a reference to determine whether the blood sugar level of the patient trends toward high levels. By viewing the long-term profile of blood sugar level establishing by the accumulated measured blood sugar values, the patient can self-check whether or not the blood sugar level in his/her body is too high upon the time for measuring the blood sugar level, or trends toward high levels during the period of time for measuring the blood sugar levels.

Then, go to step 47, when the variation of the long-term profile of blood sugar level exceeds a standard, the computer system generates a warning signal to inform the patient to improve medication or take care his/her diet. As mentioned in step 46, the variation of the long-term profile of blood sugar level depends upon e.g. the change of the slope of the curve, the frequency of the strip lines of the strip chart each of which representing a measured blood sugar value exceeding the normal value, and the frequency of the blood sugar values having been marked or highlighted in the table list. When the variation of the long-term profile of blood sugar level does not exceed a standard, the computer system generates a safety signal to inform the patient his/her blood sugar level is within the normal range. The warning/safety signal can be outputted in a form of voice, pattern and text string.

The embodiments are only used to illustrate the present invention, not intended to limit the scope thereof. Many modifications of the embodiments can be made without departing from the spirit of the present invention.

What is claimed is:

1-11. (canceled)

12. A method comprising:
periodically obtaining a specimen from a patient;
detecting a response to the periodically obtained specimen by a blood sugar determining instrument;
periodically determining a blood sugar value of said patient based on said response to the periodically obtained specimen;
storing the periodically obtained blood sugar values in a memory of said blood sugar determining instrument;
downloading the periodically obtained blood sugar values from said blood sugar determining instrument in a computer system; and
displaying the periodically obtained blood sugar values in a chart on a window of said computer system so as to establish a long-term profile of blood sugar level for said patient.

13. The method of claim 12, wherein said specimen comprises one or more of the following: a blood sample and/or a sweat sample from said patient, or combinations thereof.

14. The method of claim 13, wherein said detecting a response comprises detecting a current.

15. The method of claim 12, wherein said displaying the periodically obtained blood sugar values comprises displaying one or more of the following: a curve, a strip chart, and/or a table, or combinations thereof.

16. (canceled)

17. The method of claim 12, further comprising judging a variation of said long-term profile of blood sugar level;
generating a warning signal informing said patient when the variation of said long-term profile of blood sugar level exceeds a standard; and
generating a safety signal informing said patient when the variation of said long-term profile of blood sugar level does not exceed the standard.

18. The method of claim 17, wherein judging the variation of said long-term profile of blood sugar level comprises judging a change of a slope of a curve.

19. The method of claim 17, wherein judging the variation of said long-term profile of blood sugar level comprises judging a frequency of one or more strips shown in a strip chart representing one or more blood sugar values.

20. The method of claim 17, wherein judging the variation of said long-term profile of blood sugar level comprises judging a frequency of said blood sugar values listed in a table.

21. The method of claim 17, wherein said warning signal comprises one or more of the following: a voice, a pattern, and/or a text string, or combinations thereof.

22. The method of claim 12, wherein said computer system comprises one or more of the following: a personal computer, a notebook, and/or a palm digital assistant, or combinations thereof.

23. A method, comprising:
downloading two or more periodically determined blood sugar values from a blood sugar determining instrument in a computer system;
establishing a long-term profile of blood sugar level for said patient based at least in part on said two or more periodically determined blood sugar values via said computer system; and
generating a signal informing said patient of a variation of said long-term profile of blood sugar level via said computer system.

24. The method of claim 23, wherein said signal informing said patient comprises one or more of the following: a warning signal, and/or a safety signal, or combinations thereof.

25. The method of claim 23, wherein said signal informing said patient comprises one or more of the following: a warning signal informing said patient when the variation of said long-term profile of blood sugar level exceeds a standard, and/or a safety signal informing said patient when the variation of said long-term profile of blood sugar level does not exceed the standard, or combinations thereof.

26. The method of claim 23, wherein said generating a signal informing said patient of a variation of said long-term profile of blood sugar level comprises one or more of the following: judging a change of a slope of a curve, judging a frequency of one or more strips shown in a strip chart representing one or more blood sugar values, and/or judging a frequency of said blood sugar values listed in a table, or combinations thereof.

27. The method of claim 23, wherein said signal comprises one or more of the following: a voice signal, a pattern signal, and/or a text string signal, or combinations thereof.

28. The method of claim 23, further comprising displaying said two or more periodically determined blood sugar values in a chart on a window of said computer system.

29. The method of claim 23, further comprising:
displaying said two or more periodically determined blood sugar values in a chart on a window of said computer system; and
wherein said displaying said two or more periodically determined blood sugar values comprises displaying one or more of the following: a curve, a strip chart, and/or a table, or combinations thereof.

30. An article comprising: a storage medium having stored thereon instructions, that, if executed, results in:
downloading two or more periodically determined blood sugar values from a blood sugar determining instrument in a computer system;
establishing a long-term profile of blood sugar level for said patient based at least in part on said two or more periodically determined blood sugar values via said computer system; and
generating a signal informing said patient of a variation of said long-term profile of blood sugar level via said computer system.

31. The article of claim 30, wherein said signal informing said patient comprises one or more of the following: a warning signal, and/or a safety signal, or combinations thereof.

32. The article of claim 30, wherein said signal informing said patient comprises one or more of the following: a warning signal informing said patient when the variation of said long-term profile of blood sugar level exceeds a standard, and/or a safety signal informing said patient when the variation of said long-term profile of blood sugar level does not exceed the standard, or combinations thereof.

33. The article of claim 30, wherein said generating a signal informing said patient of a variation of said long-term profile of blood sugar level comprises one or more of the following: judging a change of a slope of a curve, judging a frequency of one or more strips shown in a strip chart representing one or more blood sugar values, and/or judging a frequency of said blood sugar values listed in a table, or combinations thereof.

34. The article of claim 30, wherein said signal comprises one or more of the following: a voice signal, a pattern signal, and/or a text string signal, or combinations thereof.

35. The article of claim 30, wherein the instructions, if executed, further result in displaying said two or more periodically determined blood sugar values in a chart on a window of said computer system.

36. The article of claim 30, wherein the instructions, if executed, further result in:
displaying said two or more periodically determined blood sugar values in a chart on a window of said computer system; and
wherein said displaying said two or more periodically determined blood sugar values comprises displaying one or more of the following: a curve, a strip chart, and/or a table, or combinations thereof.

37. An apparatus, comprising:
means for downloading two or more periodically determined blood sugar values from a blood sugar determining instrument in a computer system;
means for establishing a long-term profile of blood sugar level for said patient based at least in part on said two or more periodically determined blood sugar values via said computer system; and
means for generating a signal informing said patient of a variation of said long-term profile of blood sugar level via said computer system.

38. The apparatus of claim 37, wherein said signal informing said patient comprises one or more of the following: a warning signal, and/or a safety signal, or combinations thereof.

39. The apparatus of claim 37, wherein said signal informing said patient comprises one or more of the following: a warning signal informing said patient when the variation of said long-term profile of blood sugar level exceeds a standard, and/or a safety signal informing said patient when the variation of said long-term profile of blood sugar level does not exceed the standard, or combinations thereof.

40. The apparatus of claim 37, wherein said means for generating a signal informing said patient of a variation of said long-term profile of blood sugar level comprises one or more of the following: means for judging a change of a slope of a curve, means for judging a frequency of one or more strips shown in a strip chart representing one or more blood sugar values, and/or means for judging a frequency of said blood sugar values listed in a table, or combinations thereof.

41. The apparatus of claim 37, wherein said signal comprises one or more of the following: a voice signal, a pattern signal, and/or a text string signal, or combinations thereof.

42. The apparatus of claim 37, further comprising means for displaying said two or more periodically determined blood sugar values in a chart on said computer system.

43. The apparatus of claim 37, further comprising:
means for displaying said two or more periodically determined blood sugar values in a chart on said computer system; and
wherein said means for displaying said two or more periodically determined blood sugar values comprises displaying one or more of the following: a curve, a strip chart, and/or a table, or combinations thereof.

44. A system, comprising:
a blood sugar determining instrument, said blood sugar determining instrument capable of periodically determining two or more blood sugar values of a patient; and
a computer system, said computer system capable of:
downloading two or more periodically determined blood sugar values from a blood sugar determining instrument,
establishing a long-term profile of blood sugar level for said patient based at least in part on said two or more periodically determined blood sugar, and
generating a signal informing said patient of a variation of said long-term profile of blood sugar level.

45. The system of claim 44, wherein said signal informing said patient comprises one or more of the following: a warning signal, and/or a safety signal, or combinations thereof.

46. The system of claim 44, wherein said signal informing said patient comprises one or more of the following: a warning signal informing said patient when the variation of said long-term profile of blood sugar level exceeds a standard, and/or a safety signal informing said patient when the variation of said long-term profile of blood sugar level does not exceed the standard, or combinations thereof.

47. The system of claim 44, wherein said computer system is further capable of generating a signal informing said patient of a variation of said long-term profile of blood sugar level comprising one or more of the following: judging a change of a slope of a curve, judging a frequency of one or more strips shown in a strip chart representing one or more blood sugar values, and/or judging a frequency of said blood sugar values listed in a table, or combinations thereof.

48. The system of claim 44, wherein said signal comprises one or more of the following: a voice signal, a pattern signal, and/or a text string signal, or combinations thereof.

49. The system of claim 44, wherein said computer system comprises a window capable of displaying said two or more periodically determined blood sugar values in a chart.

50. The system of claim 44, wherein said computer system comprises a window capable of displaying said two or more periodically determined blood sugar values in a chart, said chart comprising one or more of the following: a curve, a strip chart, and/or a table, or combinations thereof.

51. The system of claim 44, wherein said blood sugar determining instrument is further capable of determining two or more blood sugar values via one or more of the following samples comprising: a blood sample and/or a sweat sample from said patient, or combinations thereof.

52. The system of claim 44, wherein said blood sugar determining instrument is further capable of determining two or more blood sugar values based at least in part on detecting a current.
